(19) United States
(12) Patent Application Publication
(10) Pub. No.: US 2026/0090917 A1
(43) Pub. Date: Apr. 2, 2026

(54) LASER ENERGY CONTROL INTERWORKING WITH LASER SYSTEM

(71) Applicant: Alcon Inc., Fribourg (CH)

(72) Inventor: David Jung, Tustin, CA (US)

(21) Appl. No.: 19/329,983

(22) Filed: Sep. 16, 2025

Related U.S. Application Data
(60) Provisional application No. 63/699,889, filed on Sep. 27, 2024.

(51) Int. Cl. A61F 9/008

(52) U.S. Cl. CPC A61F 9/00825; A61F 2009/0087; A61F 2009/00887; A61F 2009/00891

(57) ABSTRACT

A system includes a laser system and a controller. The laser system includes a laser configured to emit electromagnetic radiation in laser pulses. The controller is configured to obtain an indication of an interworking control scheme for communicating with the laser system. The controller is also configured to receive inputs from an input device. The controller is further configured to communicate with the laser system for control of the laser based on the inputs and according to the interworking control scheme.

### INTRODUCTION

Lasers are used in many different medical procedures including a number of different ophthalmic procedures. For example, lasers may be used in cataract surgery, such as for fragmenting the cataractous lens. In some procedures, a laser is used for initial fragmentation of the lens, followed by phacoemulsification of the lens by an ultrasonic handpiece to complete the breakdown of the lens for removal. In other procedures, the laser may be used for complete fragmentation and/or phacoemulsification of the lens for removal, without the need for a separate application of ultrasonic energy. Lasers may also be used for other steps in cataract surgery, such as for making corneal incision(s) and/or opening the capsule.

Lasers may also be used in glaucoma surgery. For example, a laser may be used to form all or part of a channel through the trabecular meshwork or scleral tissue for drainage of aqueous humor from the eye.

Lasers may also be used in vitreoretinal surgery. In some procedures, a laser may be used for vitrectomy, to sever or break the vitreous fibers for removal. The laser may be incorporated into a vitrectomy probe, and the energy from the laser may be applied to the vitreous fibers to sever or break the vitreous fibers for removal.

In other vitreoretinal applications, lasers may be used for photocoagulation of retinal tissue. Laser photocoagulation may be used to treat issues such as retinal tears and/or the effects of diabetic retinopathy.

### SUMMARY

In certain embodiments, a system for ophthalmic procedures is provided. The system includes a laser system and a controller. The controller is configured to obtain an indication of an interworking control scheme for communicating with the laser system. The controller is also configured to receive inputs from an input device. The controller is further configured to communicate with the laser system for control of the laser based on the inputs and according to the interworking control scheme.

In certain embodiments, a method is provided that includes obtaining an indication of an interworking control scheme for communicating with a laser system comprising a laser configured to emit electromagnetic radiation in laser pulses. The method also includes receiving inputs from an input device. The method further includes communicating with the laser system for control of the laser based on the inputs and according to the interworking control scheme.

In certain embodiments, an apparatus is provided. The apparatus includes one or more memories collectively storing instructions, and one or more processors coupled to the one or more memories. The one or more processors are collectively configured to execute the instructions to cause the apparatus to perform an operation. The operation includes obtaining an indication of an interworking control scheme for communicating with a laser system comprising a laser configured to emit electromagnetic radiation in laser pulses. The operation also includes receiving inputs from an input device. The operation further includes communicating with the laser system for control of the laser based on the inputs and according to the interworking control scheme.

### BRIEF DESCRIPTION OF THE DRAWINGS

The drawings described herein are for illustrative purposes only, are schematic in nature, and are intended to be exemplary rather than to limit the scope of the disclosure.

FIG. 1 illustrates an example surgical console of a surgical system, according to certain embodiments described herein.

FIG. 2 illustrates an example surgical system including a laser system, according to certain embodiments described herein.

FIG. 3 illustrates an example architecture of an energy controller, according to certain embodiments described herein.

FIG. 4 illustrates an example architecture of an external laser control module within the energy controller illustrated in FIG. 3, according to certain embodiments described herein.

FIG. 5 illustrates an example interworking control message, according to certain embodiments described herein.

FIG. 6 illustrates an example of operational subranges for an input device, according to certain embodiments described herein.

FIG. 7 illustrates an example scenario for interworking with a laser system, according to certain embodiments described herein.

FIGS. 8A-8B illustrate another example scenario for interworking with a laser system, according to certain embodiments described herein.

FIG. 9 illustrates a flowchart of an example method for interworking with a laser system, according to certain embodiments described herein.

FIG. 10 illustrates an example computing system, according to certain embodiments described herein.

The above summary is not intended to represent every possible embodiment or every aspect of the subject disclosure. Rather the foregoing summary is intended to exemplify some of the novel aspects and features disclosed herein. The above features and advantages, and other features and advantages of the subject disclosure, will be readily apparent from the following detailed description of representative embodiments and modes for carrying out the subject disclosure when taken in connection with the accompanying drawings and the appended claims.

### DETAILED DESCRIPTION

The present disclosure relates to laser systems for ophthalmic (eye) procedures, and more specifically, to techniques for energy control interworking with laser systems for ophthalmic procedures.

As noted, laser systems are used in many different ophthalmic procedures. Some of these laser systems emit laser beams in pulses, with the pulses having a desired duration and repetition rate. Operating a laser in pulses can achieve desirable power and energy characteristics for a particular application. Additionally, while certain laser systems include internal energy control functionality in which the amount of energy of a laser beam is controlled by the laser system itself, in some systems, it may be desirable to control the energy and other functionality of a laser system upstream (e.g., from a different device/system). Existing systems, however, may lack functionality for efficiently interworking with external laser systems that have internal energy control functionality. Accordingly, there is a need for improved systems and techniques for energy control interworking with laser systems.

Certain embodiments herein provide techniques and systems for interworking with a laser system having internal energy control functionality. As described in greater detail herein, in certain embodiments, a system for ophthalmic procedures includes interworking functionality for controlling the energy and/or other functionality of a laser system equipped with its own energy control capabilities. In certain embodiments, the interworking functionality includes a message-based interworking control scheme for communicating with a laser system in order to perform energy control for the laser system. Additionally or alternatively, in certain embodiments, the interworking functionality includes an analog-based interworking control scheme for communicating with the laser system in order to perform energy control for the laser system.

The techniques and systems described herein for energy control interworking with a laser system provide various technical advantages. For example, in certain embodiments, the techniques described herein enable systems to efficiently interact with and control external laser systems equipped with internal energy control schemes, improving interoperability of such systems with different types of laser systems. As such, the techniques described herein can provide additional functionality for energy control while reducing overall complexity of systems for ophthalmic procedures.

As used herein, a hyphenated form of a reference numeral refers to a specific instance of an element and the un-hyphenated form of the reference numeral refers to the collective element. Thus, for example, device “12-1” refers to an instance of a device class, which may be referred to collectively as devices “12” and any one of which may be referred to generically as a device “12.” The terms “attached,” “connected,” “coupled,” and the like mean attachment, connection, coupling, etc., of one part to another either directly or indirectly through one or more other parts, unless direct or indirect attachment, connection, coupling, etc., is specified.

Although the terms “first,” “second,” “third,” etc., may be used herein to describe various devices, circuits, elements, components, regions, layers and/or sections, these devices, circuits, elements, components, regions, layers and/or sections should not be limited by these terms. These terms may be only used to distinguish one device, circuit, element, component, region, layer or section from another device, circuit, element, component, region, layer, or section. Terms such as “first,” “second,” and other numerical terms, when used herein, do not imply a sequence or order unless clearly indicated by the context. Thus, a first device, circuit, element, component, region, layer, or section discussed herein could be termed a second device, circuit, element, component, region, layer, or section without departing from various embodiments described herein.

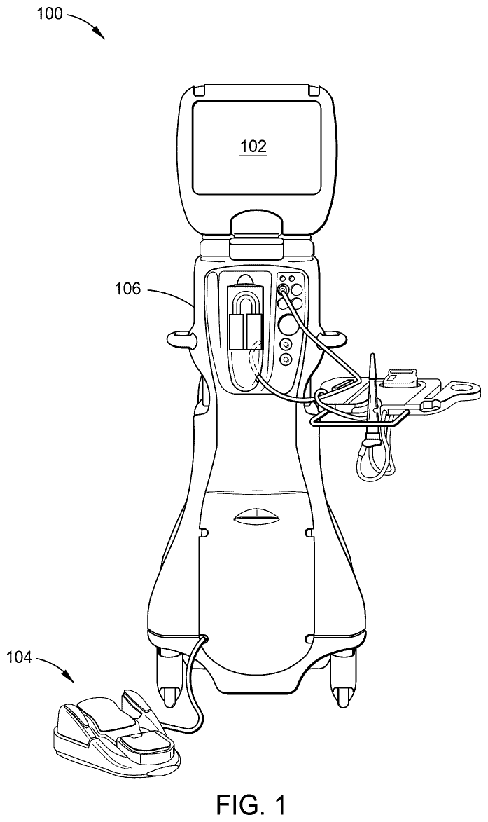

FIG. 1 illustrates an example ophthalmic surgical console 106 of a surgical system 100, according to certain embodiments described herein. As depicted in FIG. 1, the ophthalmic surgical console 106 includes a housing and a display device 102. The housing contains one or more computing systems, which control the functionality of one or more systems of the surgical console 106. For example, the surgical console 106 may include a fluidics system that includes an irrigation system for delivering fluid to an eye and an aspiration system for aspirating fluid from the eye. The display device 102 may display information relating to system operation and performance during an ophthalmic surgical procedure. The display device 102 can also include a user interface such that interactions with user interface elements of the user interface cause the one or more computer systems to modify functionality provided by at least one system of the surgical console 106 that may be used in performing an ophthalmic surgical procedure.

The surgical system 100 also includes an input device 104, which is illustrated as a footswitch. The input device 104 can be communicatively coupled to the surgical console 106 via a wired or wireless connection. The input device 104 may be an adjustable input device that an operator may actuate over an operating range for controlling one or more functions. In the illustrated example in which the input device 104 is a footswitch, the footswitch can be pressed downward to various positions over the operating range to control functionality as described further herein. While a footswitch is shown, the input device 104 can include other adjustable input devices, such as hand-operated buttons or knobs.

An example surgical system 100 in accordance with this disclosure may include a laser system suitable for one or more ophthalmic procedures. In certain embodiments described herein, the surgical system 100 may include interworking functionality for controlling the energy and/or other functionality of such a laser system, which may or may not include an internal energy control scheme.

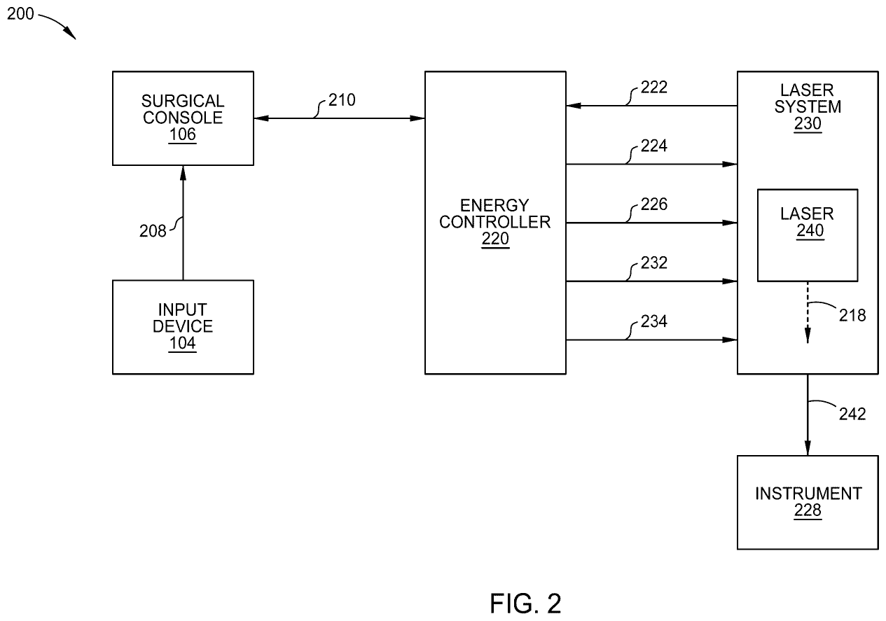

FIG. 2 illustrates an example surgical system 200 including a laser system 230, according to certain embodiments described herein. Note the surgical system 200 may be an illustrative example implementation of the surgical system 100 illustrated in FIG. 1. As shown, the surgical system 200 includes, without limitation, a surgical console 106, an input device 104, an energy controller 220, a laser system 230, and an instrument 228.

In certain embodiments, the energy controller 220 is communicatively coupled to the surgical console 106 via a connection 210, which may be a wired connection or a wireless connection. The energy controller 220 may receive information (e.g., inputs or messages) transmitted from the surgical console 106 via the connection 210, and the energy controller 220 may also transmit information (e.g., confirmations or acknowledgments) to the surgical console 106 via the connection 210. In certain embodiments, the input device 104 is communicatively coupled to the surgical console 106 via a connection 208, which may be a wired connection or a wireless connection. The input device 104 may transmit information (e.g., inputs) to the surgical console 106 via the connection 208.

In certain embodiments, the laser system 230 includes a laser 240. Although not shown, in addition to the laser 240, the laser system 230 may have other components. For example, the laser system 230 may include components for operating the laser 240, such as a power supply, laser pumps, laser energy control, and monitor, as illustrative, non-limiting examples. In addition, the laser system 230 may include other components in the optical path of a laser(s) output from the laser 240, such as one or more lenses, mirrors, and optical fibers, as illustrative, non-limiting examples. The laser system 230 is generally designed to direct laser electromagnetic radiation from the laser 240 to an output port. The laser system 230 may direct the laser electromagnetic radiation from the laser 240 to the output port through one or more optical components, such as lenses and mirrors, as illustrative examples.

An instrument 228 may be optically connected to the laser system 230 to receive the laser electromagnetic radiation from the output port. The instrument 228 may be, for example, a handpiece for an ophthalmic procedure. The instrument 228 may be connected to the laser system 230 by a delivery optical fiber 242. The delivery optical fiber 242 may be flexible and relatively long (e.g., 6 feet, 8 feet, 10 feet, etc.) to give an operator flexibility in maneuvering the instrument 228 at some distance away from the laser system 230. The laser electromagnetic radiation may be transmitted from the laser system 230, through the delivery optical fiber 242 and the instrument 228, and from an output tip of the instrument 228 to the desired target, such as a lens or lens fragment in the eye of a patient.

In certain embodiments, the laser system 230 may be suitable for cataract surgery. In certain embodiments, the output energy of the laser system 230 is suitable for fragmentation and/or emulsification of a cataractous lens. In some examples, the laser output is used for fragmentation and/or phacoemulsification of the lens to a sufficient degree for removal of the lens.

In certain embodiments, the laser system 230 may be suitable for glaucoma surgery. In certain embodiments, the output energy of the laser system 230 is suitable for making or facilitating the formation of a drainage channel in eye tissue. In some examples, the drainage channel is configured to reduce intraocular pressure.

The laser 240 may be any type of laser suitable for the desired application. The laser 240 may output suitable electromagnetic radiation at any suitable wavelength. For example, the laser 240 may emit electromagnetic radiation in one or more wavelengths in the visible, infrared, and/or ultraviolet wavelengths. The laser 240 may operate or be operated to emit a continuous beam of electromagnetic radiation. Alternatively, the laser 240 may operate or be operated to emit a pulsed beam.

In one or more examples, the laser 240 operates in the infrared range. For example, the laser 240 may output electromagnetic radiation in the mid-infrared range, e.g., in a range of about 2.0 microns to about 4.0 microns. Some examples of wavelengths include about 2.5 microns to 3.5 microns, such as about 2.775 microns, about 2.8 microns, or about 3.0 microns. Such a laser may be suitable, for example, for lens fragmentation in cataract surgery, or for other procedures.

In one or more embodiments, the energy controller 220 receives a laser trigger 222 from the laser system 230. In certain embodiments, the energy controller 220 may process the laser trigger 222 to control an amount of electromagnetic radiation included in laser pulses output from the laser system 230 via a laser power control 224. In addition to controlling the amount of electromagnetic radiation included in laser pulses output from the laser system 230, the energy controller 220 can process the laser trigger 222 to control timing and other characteristics (e.g., frequency, duty ratio, etc.) of the laser pulses output from the laser system 230 via a pulse picking control 226. For example, the energy controller 220, via the pulse picking control 226, may selectively perform passing and blocking of laser pulses (e.g., laser pulse train 218) in order to control the timing and other characteristics of the laser pulses output from the laser system 230. The laser power control 224 and/or pulse picking control 226 may be based on inputs to the surgical system 200 (e.g., inputs to the surgical console 106), including from the input device 104, if provided. In some embodiments, the laser power control 224 and/or pulse picking control 226 may be based on inputs provided via the instrument 228.

In certain embodiments, the laser system 230 may be housed within the surgical console 106. In certain other embodiments, the laser system 230 may be housed in a separate console that communicates with the surgical console 106. In yet certain other embodiments, one or more parts (or components) of the laser system 230, such as the laser 240, may be housed in a separate console that communicates with the surgical console 106, and one or more other parts of the laser system 230 may be housed in the surgical console 106. In yet certain other embodiments, the laser system 230 may be in a stand-alone housing that receives inputs from the input device 104 without the need for a separate surgical console 106.

Notwithstanding whether the laser system 230 is housed within the surgical console 106, within a separate console that communicates with the surgical console 106, or partially within the surgical console 106, in certain cases, the laser system 230 may include an internal energy controller for controlling the energy and/or other characteristics (e.g., timing, frequency, duty ratio, among others) of the laser pulses output from the laser system 230. In such cases, the laser system 230 may be referred to as an “external” laser system 230 in that it is equipped with its own internal energy control scheme, even if some or all of the laser system 230 may be housed within the surgical console 106.

As noted, in surgical systems that include such “external” laser systems, there may be interoperability issues with interworking with the “external” laser systems to control the energy and/or other characteristics of the laser pulses output from the “external” laser systems (e.g., via inputs to the surgical console 106, such as from the input device 104). Accordingly, to address the deficiencies above, certain embodiments herein provide surgical system 200, which is equipped with an energy controller 220, which is configured with various interworking functionality for communicating with the laser system 230 in order to perform energy control for the laser system 230 (e.g., via inputs to the surgical console 106, such as from the input device 104). In certain embodiments, the energy controller 220 may support a message-based interworking control scheme for communicating with the laser system 230, an analog-based interworking control scheme for communicating with the laser system 230, or a combination thereof.

In certain embodiments, when the message-based interworking control scheme is selected (e.g., via the surgical console 106), the energy controller 220 may receive inputs for controlling the laser system 230 via the surgical console 106 and/or input device 104, generate a message control 232 (including one or more messages) including control information based on the inputs, and provide the message control 232 to the laser system 230. As such, the message-based interworking control scheme may enable the laser system 230 to perform energy control, pulse picking, and/or other functions based on the control information provided via the message control 232.

In certain embodiments, when the analog-based interworking control scheme is selected (e.g., via the surgical console 106), the energy controller 220 may receive inputs for controlling the laser system 230 via the surgical console 106 and/or input device 104, generate analog control signals 234 for controlling the amount of energy, pulse picking, and/other functions of the laser system 230 based on the inputs, and send the analog control signals 234 to the laser system 230. As described herein, the analog control signals 234 may be generated using any suitable analog conversion techniques, such as pulse width modulation (PWM) and serial peripheral interface (SPI), as illustrative examples.

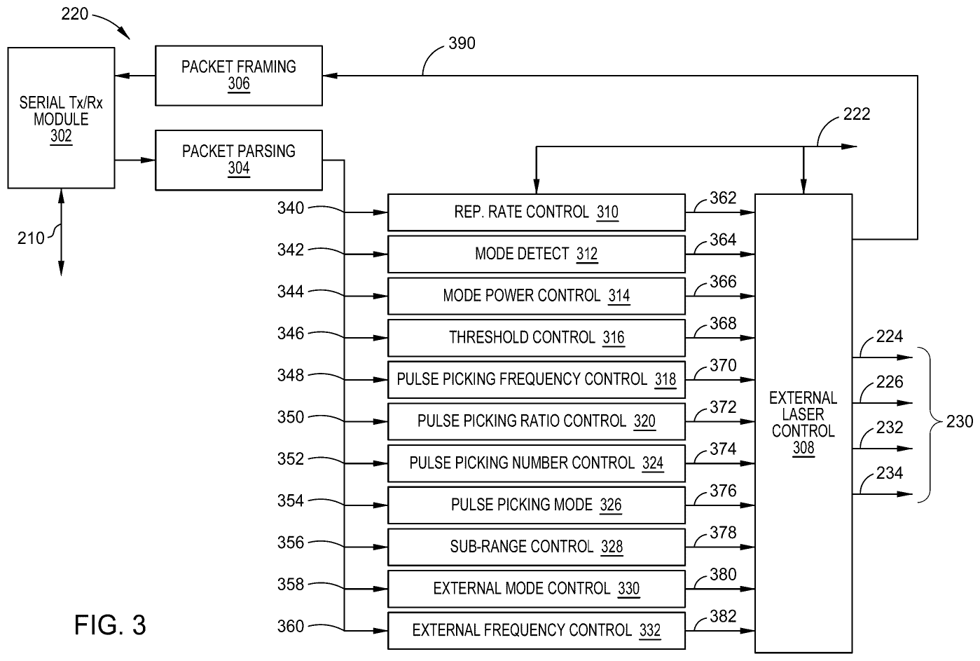

FIG. 3 illustrates an example architecture of the energy controller 220 illustrated in FIG. 2, according to certain embodiments described herein. As depicted in FIG. 3, the energy controller 220 includes a serial transmitter/receiver (Tx/Rx) module 302, a packet parsing module 304, a packet framing module 306, and an external laser control module 308. In certain embodiments, the energy controller 220 may also include a repetition rate control module 310, a mode detect module 312, a mode power control module 314, a threshold control module 316, a pulse picking frequency control module 318, a pulse picking ratio control module 320, a pulse picking number control module 324, a pulse picking mode module 326, a subrange control module 328, an external mode control module 330, and an external frequency control module 332.

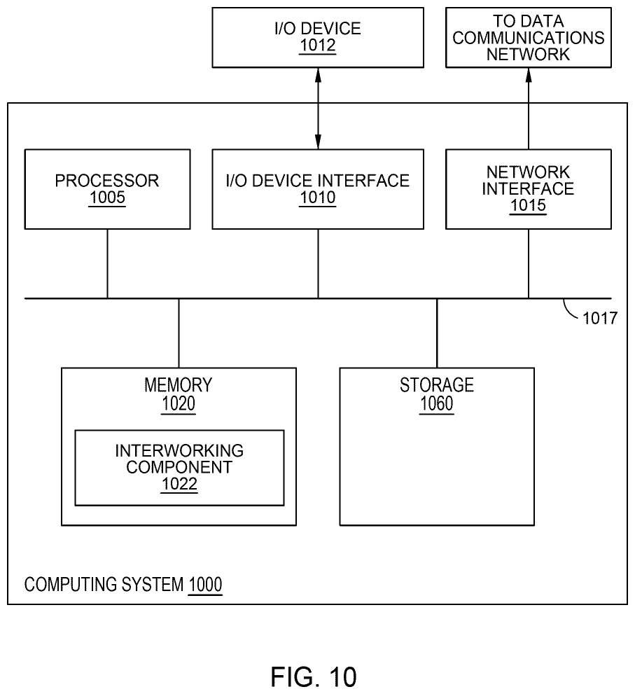

Although modules 302, 304, 306, 308, 310, 312, 314, 316, 318, 320 324, 326, 328, 330, and 332 (collectively referred to as modules 302-332) are described as distinct modules, it is appreciated that the energy controller 220 may include additional modules or fewer modules than illustrated in FIG. 3. It is also to be appreciated that each of the modules 302-332 may be implemented using software, firmware, hardware, or any combination thereof. For example, in certain embodiments, the modules 302-332 or any combination thereof may be implemented using compute resources, such as processors, memory, storage, and the like, such as those illustrated within computing system 1000 in FIG. 10.

In one or more embodiments, the serial Tx/Rx module 302 receives an input transmitted from the surgical console 106 (e.g., from the input device 104) via the connection 210. For example, the input is a message or a packet of data as described further below. In some embodiments, the serial Tx/Rx module 302 communicates the input received via the connection 210 to the packet parsing module 304. The packet parsing module 304 receives and processes the input, and the packet parsing module 304 may parse or segment the input into repetition rate data 340, mode data 342, power data 344, power threshold data 346, pulse picking frequency data 348, pulse picking duty ratio data 350, pulse picking number data 352, pulse picking mode data 354, subrange data 356, external mode data 358, and external frequency data 360.

In certain embodiments, the repetition rate control module 310 receives and processes the repetition rate data 340 in order to extract a repetition rate 362 described by the repetition rate data 340. The repetition rate may indicate a laser pulse rate for the laser 240 (e.g., 1 kilohertz (kHz), 1.5 kHz, etc.). The repetition rate control module 310 can communicate the repetition rate 362 to the external laser control module 308.

In certain embodiments, the mode detect module 312 receives and processes the mode data 342 in order to communicate a mode 364 (e.g., a sculpt mode, a quad mode, etc.) described by the mode data 342 to the external laser control module 308.

In certain embodiments, the mode power control module 314 receives and processes the power data 344 and extracts a power setting 366 (e.g., 0 percent to 100 percent) described by the power data 344. The mode power control module 314 may communicate the power setting 366 to the external laser control module 308.

In certain embodiments, the threshold control module 316 receives and processes the power threshold data 346 and extracts a maximum power setting 368 described by the power threshold data 346. The threshold control module 316 may communicate the maximum power setting 368 to the external laser control module 308.

In certain embodiments, the pulse picking frequency control module 318 receives and processes the pulse picking frequency data 348 and extracts a pulse picking frequency 370 described by the pulse picking frequency data 348. The pulse picking frequency control module 318 may communicate the pulse picking frequency 370 to the external laser control module 308.

In certain embodiments, the pulse picking ratio control module 320 receives and processes the pulse picking duty ratio data 350 and extracts a pulse picking duty ratio 372 (e.g., 1 percent to 100 percent) described by the pulse picking duty ratio data 350. The pulse picking ratio control module 320 may communicate the pulse picking duty ratio 372 to the external laser control module 308.

In certain embodiments, the pulse picking number control module 324 receives and processes the pulse picking number data 352 and extracts a pulse picking number 374 described by the pulse picking number data 352. The pulse picking number 374, for example, may indicate a number of laser pulses to be included in each pulse picking cycle. The pulse picking number control module 324 may communicate the pulse picking number 374 to the external laser control module 308.

In certain embodiments, the pulse picking mode module 326 receives and processes the pulse picking mode data 354 to extract a pulse picking mode 376 (e.g., normal mode, burst mode, etc.) described by the pulse picking mode data 354. In normal mode, the pulse picking may be based on a timer that is synchronous with the laser trigger 222. In burst mode, the pulse picking may be based on a timer that is asynchronous with respect to the laser trigger 222 and the communication message. The pulse picking mode module 326 may communicate the pulse picking mode 376 to the external laser control module 308.

In certain embodiments, the subrange control module 328 receives the subrange data 356 as describing a subrange 378 (e.g., SubRange1, SubRange2, SubRange3, etc.). In an illustrative example in which the input device 104 is a footswitch, the footswitch can be pressed downward to various positions (e.g., subranges) in order to specify the subrange 378 described by the subrange data 356 as described further below. The subrange control module 328 can process the subrange data 356 in order to communicate the subrange 378 to the external laser control module 308.

In certain embodiments, the external mode control module 330 receives and processes the external mode data 358 to extract an external mode 380 described by the external mode data 358. The external mode 380 may indicate a type of interworking control scheme for communicating with the laser system 230. As noted, the interworking control scheme may be selected from multiple types of interworking control schemes, such as message-based interworking control scheme, analog-based interworking control scheme (e.g., PWM, SPI, etc.), among others. The external mode control module 330 may communicate the external mode 380 to the external laser control module 308.

In certain embodiments, the external frequency control module 332 receives and processes the external frequency data 360 to extract an external frequency 382 described by the external frequency data 360. The external frequency may include an indication of how often the laser system 230 should be updated with external control information according to the external mode 380. The external frequency control module 332 may communicate the external frequency 382 to the external laser control module 308.

As illustrated in FIG. 3, the external laser control module 308 receives the repetition rate 362, the mode 364, the power setting 366, the maximum power setting 368, the pulse picking frequency 370, the pulse picking duty ratio 372, the pulse picking number 374, the pulse picking mode 376, the subrange 378, the external mode 380, and the external frequency 382 as inputs. In certain embodiments, the external laser control module 308 also receives the laser trigger 222 as one of the inputs.

In certain embodiments, the external laser control module 308 processes the inputs received in order to generate outputs which may include a laser power control 224, a pulse picking control 226, a message control 232, analog control signals 234, or any combination thereof. In one or more embodiments, the external laser control module 308 communicates the laser power control 224, the pulse picking control 226, the message control 232, and the analog control signals 234 to the laser system 230.

In certain embodiments, the external laser control module 308 communicates a message confirmation 390 to the packet framing module 306. In certain embodiments, the packet framing module 306 receives and processes the message confirmation 390 to format data included in the message confirmation 390 in a format which may be similar to the format of the inputs received by the packet parsing module 304. In an illustrative example, the packet framing module 306 formats the message confirmation 390 as packets of data for communication to the serial Tx/Rx module 302. In one or more examples, the serial Tx/Rx module 302 communicates data included in the message confirmation 390 to the surgical console 106 via the connection 210.

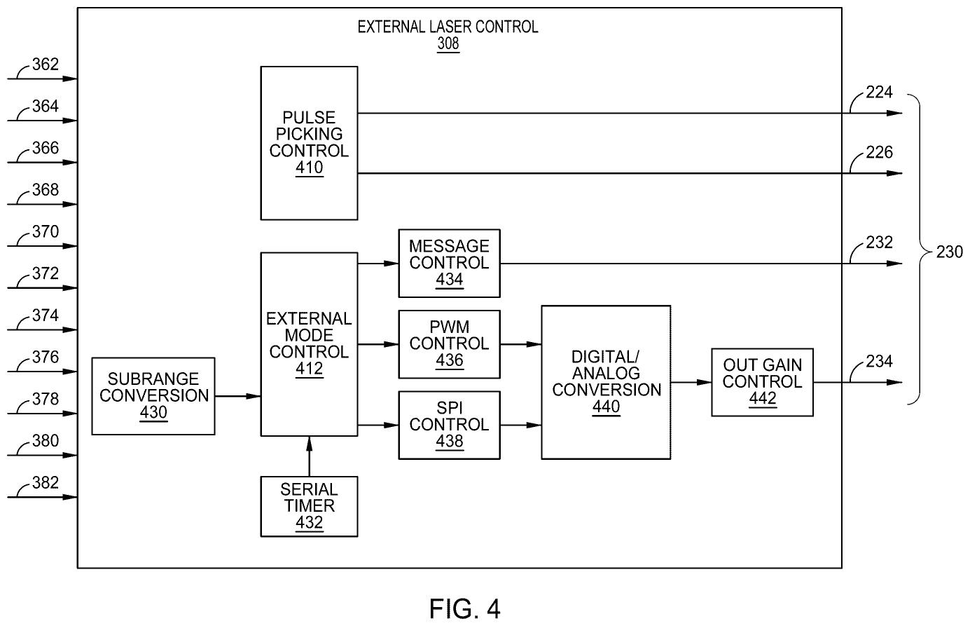

FIG. 4 illustrates an example architecture of the external laser control module 308 illustrated in FIG. 3, according to certain embodiments described herein. As shown in FIG. 4, the external laser control module 308 includes a pulse picking control module 410, an external mode control module 412, a subrange conversion module 430, a serial timer module 432, a message control module 434, a PWM control module 436, an SPI control module 438, a digital/analog conversion module 440, and an out gain control module 442.

Although modules 410, 412, 430, 432, 434, 436, 438, 440, and 442 (collectively referred to as modules 410-442) are described as distinct modules, it is to be appreciated that the external laser control module 308 may include additional modules or fewer modules than illustrated in FIG. 4. It is also to be appreciated that each of the modules 410-442 may be implemented using software, firmware, hardware, or any combination of thereof. For example, in certain embodiments, the modules 410-442 or any combination thereof may be implemented using compute resources, such as processors, memory, storage, and the like, such as those illustrated within computing system 1000 in FIG. 10.

In certain embodiments, the pulse picking control module 410 receives the repetition rate 362, the mode 364, the power setting 366, the maximum power setting 368, the pulse picking frequency 370, the pulse picking duty ratio 372, the pulse picking number 374, the pulse picking mode 376, or any combination thereof, as inputs. The pulse picking control module 410 may process the inputs to generate the laser power control 224 and the pulse picking control 226. The pulse picking control module 410 may communicate the laser power control 224 and the pulse picking control 226 to the laser system 230.

In certain embodiments, the subrange conversion module 430 receives the subrange 378 and the external mode 380. The subrange conversion module 430 may latch the subrange 378 and communicate the subrange 378 to the external mode control module 412 when the external mode 380 indicates an analog interworking control scheme (e.g., PWM, SPI, etc.). Additionally, in certain embodiments, the serial timer module 432 receives the external frequency 382 and the external mode 380. The serial timer module 432 may communicate the external frequency 382 to the external mode control module 412.

In certain embodiments, the external mode control module 412 receives the repetition rate 362, the mode 364, the power setting 366, the maximum power setting 368, the pulse picking frequency 370, the pulse picking duty ratio 372, the pulse picking number 374, the pulse picking mode 376, the subrange 378, the external mode 380, the external frequency 382, or any combination thereof, as inputs. In certain embodiments, when the external mode 380 indicates a message-based interworking control scheme, the external mode control module 412 may activate the message control module 434 and provide the inputs to the message control module 434. The message control module 434 may generate an interworking control message including the inputs (or a combination thereof) and provide the interworking control message to the laser system 230 via message control 232. The laser system 230 may then perform message decoding (e.g., decoding of the message received via message control 232), message handling, energy control, pulse picking, and other functions, based on the message control 232.

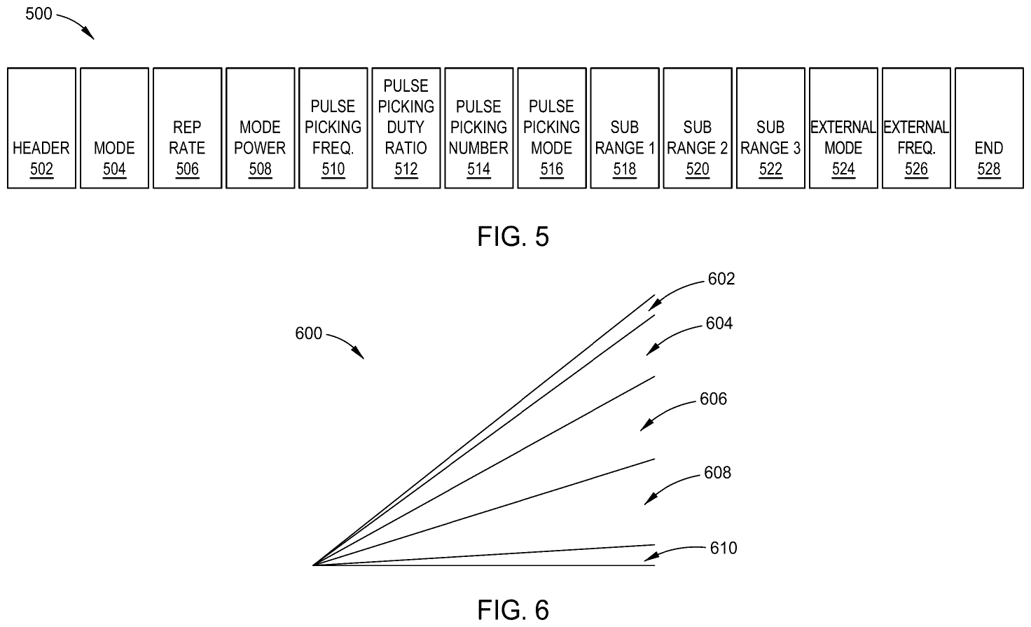

By way of example, FIG. 5 illustrates an example of an interworking control message 500, according to certain embodiments described herein. In certain embodiments, the interworking control message 500 is an illustrative example message that may be obtained by the energy controller 220. Additionally or alternatively, the interworking control message 500 is an illustrative example message that may be communicated to the laser system 230 via message control 232.

As shown, the message 500 includes packets of instructions, fields, and/or data which are organized in a format that may begin with a header 502. In one or more embodiments, the serial Tx/Rx module 302 can receive the message 500 via the connection 210 from the surgical console 106. In an example, the packet parsing module 304 processes the message 500 to identify the header 502 which indicates the beginning of the message 500. In another example, the message control module 434 may generate the message 500 by including one or more of the fields depicted in FIG. 5 beginning with the header 502.

In certain embodiments, a mode field 504 follows the header 502, and the mode field 504 indicates a selected mode of operation for the laser system 230. For example, the mode field 504 may include the mode 364. Examples of the mode 364 include a sculpting mode and a quad mode, among other modes.

In certain embodiments, a repetition rate field 506 follows the mode field 504. The repetition rate field 506 may define a selected base repetition rate for the laser 240. For example, the repetition rate field 506 may include the repetition rate 362, which defines a rate of laser pulses to be emitted from the laser 240. Examples of the repetition rate 362 include 1 kHz (1000 laser pulses per second), 1.5 kHz (1500 laser pulses per second), a frequency between 1 and 1.5 kHz, etc.

In certain embodiments, a mode power field 508 follows the repetition rate field 506, and the mode power field 508 indicates an amount of electromagnetic radiation to be included in laser pulses output from the laser system 230. For example, the mode power field 508 may include the power setting 366 and/or the maximum power setting 368.

In certain embodiments, a pulse picking frequency field 510 follows the mode power field 508. The pulse picking frequency field 510 may define a selected length of a pulse picking cycle. For example, the pulse picking frequency field 510 may include the pulse picking frequency 370. In certain embodiments, the pulse picking frequency 370 may be related to the repetition rate 362. For example, in certain cases, the pulse picking frequency 370 may not be greater than the repetition rate 362. Examples of the pulse picking frequency 370 can include 1 Hz (hertz), 500 Hz, a frequency between 1 and 500 Hz, a frequency less than 1 Hz, and so forth.

In certain embodiments, a pulse picking duty ratio field 512 follows the pulse picking frequency field 510. In various examples, the pulse picking duty ratio field 512 indicates a relative amount of time during a pulse picking cycle that laser pulses are emitted from the laser system 230 (e.g., 1 to 100 percent). In some examples, the pulse picking duty ratio field 512 may include the pulse picking duty ratio 372, which may correspond to a number of laser pulses included in a pulse picking cycle.

In certain embodiments, a pulse picking number field 514 follows the pulse picking duty ratio field 512. In one example, the pulse picking number field 514 may indicate a selected number of laser pulses to be emitted by the laser system 230 during a pulse picking cycle. In another example, the pulse picking number field 514 may indicate a maximum number of laser pulses to be emitted by the laser system 230 during a pulse picking cycle. In some cases, the pulse picking number field 514 may include the pulse picking number 374.

In certain embodiments, a pulse picking mode field 516 follows the pulse picking number field 514. The pulse picking mode field 516 can indicate a type of pulse picking mode which has been selected (e.g., a normal mode, a burst mode, etc.). In certain cases, the pulse picking mode field 516 may include the pulse picking mode 376. In one or more examples, the mode field 504 can be set to a quad mode if the pulse picking mode field 516 indicates that a type of pulse picking mode has been selected. In certain embodiments, if the pulse picking mode field 516 indicates a normal mode, then a synchronous firing timer (e.g., synchronous with the laser trigger 222) is selected. In one or more embodiments, if the pulse picking mode field 516 indicates a burst mode, then an asynchronous firing timer (e.g., asynchronous with the laser trigger 222 and synchronous with the message 500) is selected.

In one or more embodiments, a SubRange1 field 518 follows the pulse picking mode field 516. In examples in which the input device 104 is a footswitch, the input device 104 is adjustable within a first subrange, and the SubRange1 field 518 can indicate a position of a pedal of the input device 104 within the first subrange (e.g., 0 to 100). For example, if the SubRange1 field 518 has a value of 0, then the pedal has not actuated into the first subrange. In some embodiments, if the SubRange1 field 518 has a value of 100, then the pedal has actuated completely through the first subrange. In an example, if the SubRange1 field 518 has a value of 50, then the pedal is actuated halfway through the first subrange. In some embodiments, the first subrange is a first pre-active range (e.g., actuation of the pedal of the input device 104 within the first subrange does not energize the laser 240).

In certain embodiments, a SubRange2 field 520 follows the SubRange1 field 518. The SubRange2 field 520 indicates a position of the pedal of the input device 104 within a second subrange (e.g., 0 to 100). Similar to the SubRange1 field 518, if the SubRange2 field 520 has a value of 0, then the pedal has not actuated into the second subrange. If the Subrange2 field 520 has a value of 100, then the pedal has actuated completely through the second subrange. In one or more embodiments, the second subrange is a second pre-active range (e.g., actuation of the pedal of the input device 104 within the second subrange does not energize the laser 240).

In some embodiments, a SubRange3 field 522 follows the SubRange2 field 520 in the message 500. For example, the SubRange3 field 522 indicates a position of the pedal of the input device 104 within a third subrange (e.g., 0 to 100). In an example, the third subrange is an active subrange, and an actuation of the pedal of the input device 104 within the third subrange energizes the laser 240. In other examples, one or more additional operational subranges may follow the SubRange3 field 522. For example, a SubRange4 field, SubRange5 field, SubRange6, and so on, may follow the SubRange3 field 522.

In some embodiments, an external mode field 524 follows the SubRange3 field 522. The external mode field 524 may indicate a type of interworking control scheme. For instance, the external mode field 524 may include the external mode 380. Examples of interworking control schemes may include a message-based interworking control scheme and an analog interworking control scheme (e.g., PWM, SPI, etc.), as illustrative examples.

In some embodiments, an external frequency field 526 follows the external mode field 524. The external frequency field 526 may include the external frequency 382, which may define a selected frequency for updating the laser system 230 with external control information. Examples of the external frequency 382 can include 1 kHz, 100 kHz, a frequency between 1 and 100 kHz, and so forth.

In one or more embodiments, an end field 528 follows the external frequency field 526. For example, the packet parsing module 304 may process the message 500 to identify the end field 528 which indicates an end of the message 500. In another example, the message control module 434 may include the end field 528 within the message 500 so that the laser system 230 can identify the end of the message 500.

Referring back to FIG. 4, in certain embodiments, when the external mode 380 indicates an analog-based interworking control scheme, such as PWM control, the external mode control module 412 may activate PWM control module 436. The PWM control module 436 may obtain the latched subrange data from the subrange conversion module 430 and an indication of the external frequency 382 via the serial timer module 432. For example, the serial timer module 432 may provide a clock frequency with the PWM control module 436. The PWM control module 436 may transform the subrange data to a PWM signal per serial frequency, and provide the PWM signals to the digital/analog conversion module 440. The digital/analog conversion module 440 may convert the digital serial data to analog form, and send the analog data to the out gain control module 442. The out gain control module 442 may adjust the analog output up to an input range of the laser system 230, and provide the analog control signal 234 to the laser system 230.

In certain embodiments, when the external mode 380 indicates an analog interworking control scheme, such as SPI control, the external mode control module 412 may activate SPI control module 438. The SPI control module 438 may obtain the latched subrange data from the subrange conversion module 430 and an indication of the external frequency 382 via the serial timer module 432. For example, the serial timer module 432 may provide a clock frequency with the SPI control module 438. The SPI control module 438 may transform the subrange data to a set of SPI signals, and provide the SPI signals to the digital/analog conversion module 440. The digital/analog conversion module 440 may convert the digital serial data to analog form, and send the analog data to the out gain control module 442. The out gain control module 442 may adjust the analog output up to an input range of the laser system 230, and provide the analog control signal 234 to the laser system 230.

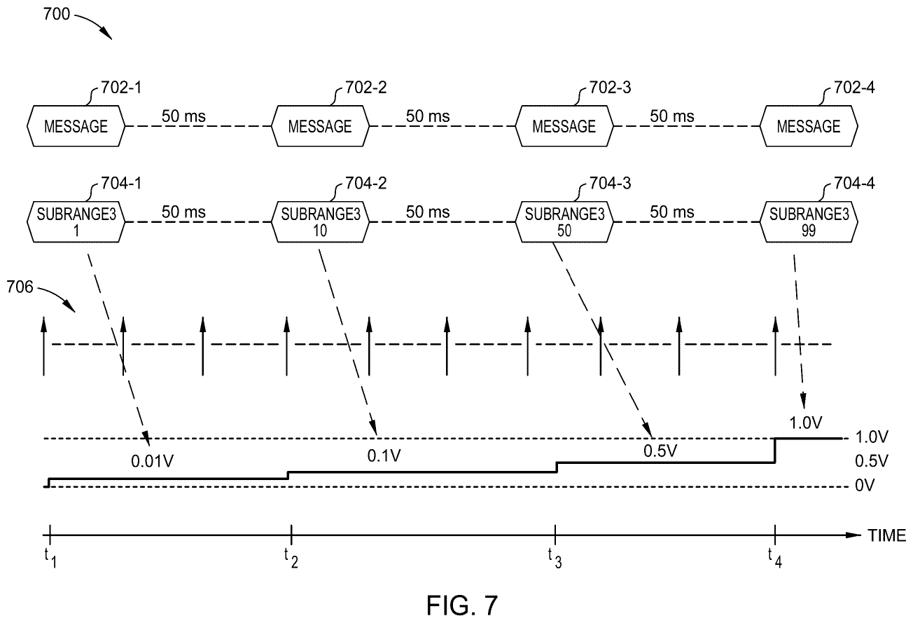

FIG. 6 illustrates an example of operational subranges 600 for an input device, such as input device 104, according to embodiments described herein. As depicted in FIG. 6, the operational subranges 600 include a range zero 602, a range one 604, a range two 606, a range three 608, and a bottom range 610. As described above, the pedal of the input device 104 can actuate within the range one 604 (the first subrange), the range two 606 (the second subrange), and the range three 608 (the third subrange). In certain embodiments, an actuation of the pedal of the input device 104 into the range one 604 activates the surgical console 106 for a specific function, such as irrigation, without energizing the laser 240. In some embodiments, an actuation of the pedal of the input device 104 into the range two 606 activates the surgical console 106 for a different specific function, such as aspiration, without energizing the laser 240. For example, the irrigation function may or may not continue operation while the pedal of the input device 104 is actuated within the range two 606. In one or more embodiments, an actuation of the pedal of the input device 104 into the range three 608 energizes the laser 240. In various examples, the irrigation and/or the aspiration may or may not continue operation while the pedal of the input device 104 is actuated into the range three 608. By adjusting or actuating the pedal of the input device 104 within the range three 608, an operator may dynamically adjust output from the laser system 230. In some embodiments, adjusting or actuating the pedal of the input device 104 within the range three 608 specifies a value of the SubRange3 field 522 which can be used to specify values of the mode field 504, the repetition rate field 506, the mode power field 508, the pulse picking frequency field 510, the pulse picking duty ratio field 512, the pulse picking number field 514, the pulse picking mode field 516, or any combination thereof. Note that although three operational subranges are shown, an input device as described herein may have more or less operational subranges.

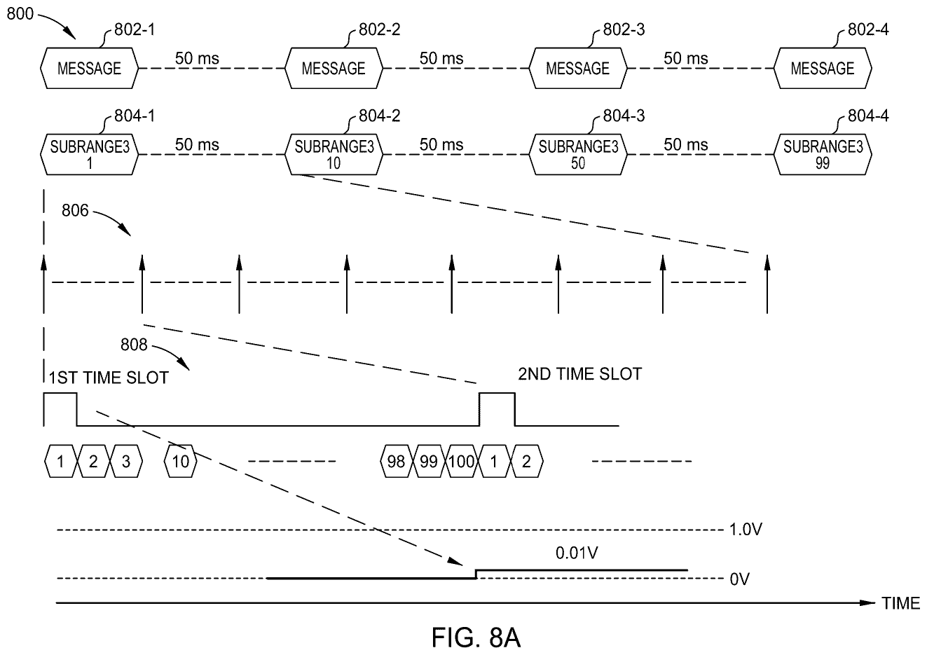

FIG. 7 depicts an example scenario 700 for interworking with a laser system (e.g. laser system 230), according to certain embodiments. As shown, the external laser control module 308 may receive a message train including messages 702-1 to 702-4. Each message 702 may be similar to the interworking control message 500 illustrated in FIG. 5. In some cases, the messages 702 may be received periodically over time. Here, for example, a message 702 may be received every 50 milliseconds (ms) or some other periodic time interval. In certain embodiments, the messages 702 may be received by the external laser control module 308 via the packet parsing module 304.

In certain aspects, after parsing, the external laser control module 308 may receive a respective indication of SubRange3 information within each message 702. As depicted in FIG. 7, message 702-1 may include a value 704-1 of “1” for SubRange3 data, message 702-2 may include a value 704-2 of “10” for SubRange3 data, message 702-3 may include a value 704-3 of “50” for SubRange3 data, and message 702-4 may include a value 704-4 of “100” for SubRange 3 data.

In certain aspects, the external laser control module 308 may convert the respective SubRange3 value 704 within each message 702 into a respective analog signal. As shown in FIG. 7, for example, assuming a 1 kHz serial timer 706 (e.g., indicated via serial timer module 432), the SubRange3 value 704-1 within message 702-1 may correspond to an analog output of 0.01 volts (e.g., from t₁ to t₂), the SubRange3 value 704-2 within message 702-2 may correspond to an analog output of 0.1 volts (e.g., from t₂ to t₃), the SubRange3 value 704-3 within message 702-3 may correspond to an analog output of 0.5 volts (e.g., from t₃ to t₄), and the SubRange3 value 704-4 within message 702-4 may correspond to an analog output of 1.0 volts (e.g., from t₄).

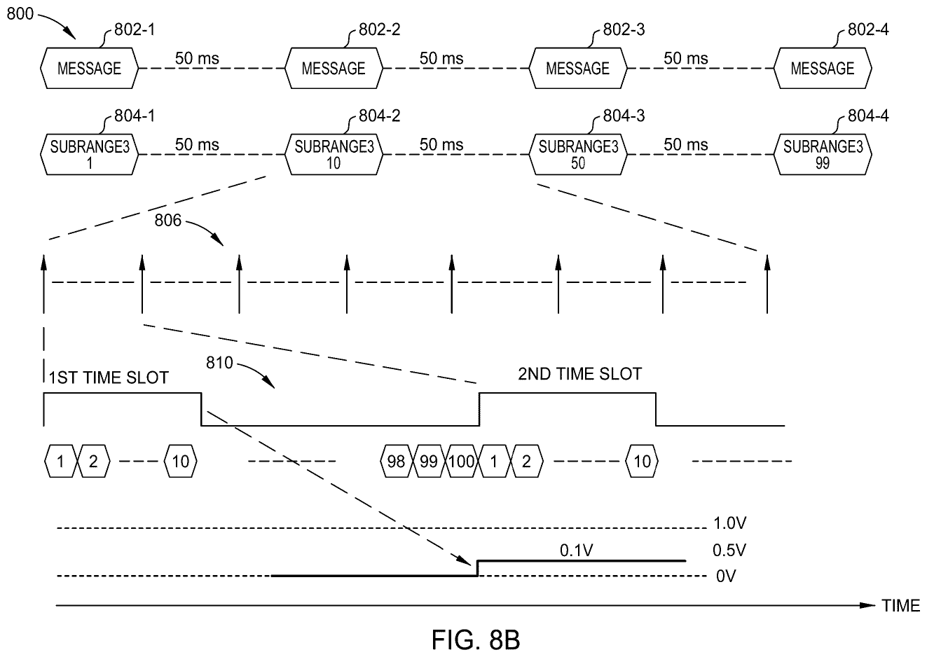

FIGS. 8A-8B depict an example scenario 800 for interworking with a laser system (e.g. laser system 230), according to certain embodiments. As shown, the external laser control module 308 may receive a message train including messages 802-1 to 802-4. Each message 802 may be similar to the interworking control message 500 illustrated in FIG. 5. In some cases, the messages 802 may be received periodically over time. Here, for example, a message 802 may be received every 50 milliseconds (ms) or some other periodic time interval. In certain embodiments, the messages 802 may be received by the external laser control module 308 via the packet parsing module 304.

In certain aspects, after parsing, the external laser control module 308 may receive a respective indication of SubRange3 information within each message 802. As depicted in FIGS. 8A-8B, message 802-1 may include a value 804-1 of “1” for SubRange3 data, message 802-2 may include a value 804-2 of “10” for SubRange3 data, message 802-3 may include a value 804-3 of “50” for SubRange3 data, and message 802-4 may include a value 804-4 of “100” for SubRange 3 data.

In certain aspects, assuming PWM has been selected as an interworking control scheme, the external laser control module 308 may generate a PWM signal based on the messages 802. By way of example, FIG. 8A depicts an example PWM signal 808 corresponding to the SubRange3 value 804-1 of message 802-1 during a 1^{st} time slot from receipt of the message 802-1, assuming a 1 kHz serial timer 806. As also depicted in FIG. 8A, for example, the PWM signal 808 has an analog output of 0.01 volts.

By way of another example, FIG. 8B depicts an example PWM signal 810 corresponding to the SubRange3 value 804-2 of message 802-2 during a 1^{st} time slot from receipt of the message 802-2, assuming a 1 kHz serial timer 806. As also depicted in FIG. 8B, for example, the PWM signal 810 has an analog output of 0.1 volts.

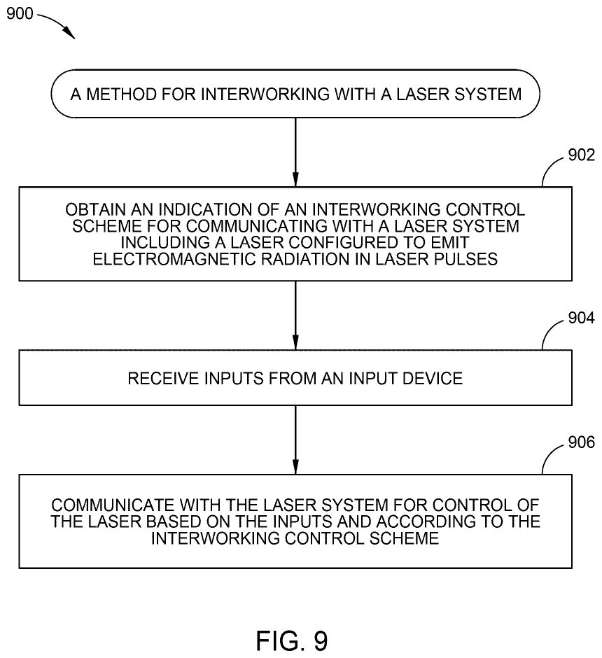

FIG. 9 is a flowchart of a method 900 for interworking with a laser system, according to certain embodiments. The method 900 may be performed by a controller (e.g., energy controller 220 or one or more components thereof, such as external laser control module 308).

Method 900 enters at block 902, where the controller obtains an indication of an interworking control scheme for communicating with a laser system (e.g., laser system 230) comprising a laser (e.g., laser 240) configured to emit electromagnetic radiation in laser pulses.

At block 904, the controller receives inputs from an input device (e.g., input device 104). In certain embodiments, the input device is an adjustable footswitch configured to actuate within subranges (e.g., subranges depicted in FIG. 6) to communicate the inputs.

At block 906, the controller communicates with the laser system for control of the laser based on the inputs and according to the interworking control scheme.

In certain embodiments, the interworking control scheme includes a message-based interworking control scheme. In such embodiments, the controller may be configured to generate a message including the inputs and transmit the message to the laser system, e.g., via message control 232. The message control 232 may trigger an internal controller within the laser system 230 to control operation of the laser based on the inputs within the message control 232.

In certain embodiments, the interworking control scheme includes an analog-based interworking control scheme, such as a PWM control scheme and SPI control scheme, as illustrative examples. In such embodiments, the controller may be configured to generate one or more control signals (e.g., analog control signals 234) and transmit the one or more control signals to the laser system.

FIG. 10 illustrates an example computing system 1000 configured to perform interworking for a laser system 230, according to certain embodiments. As shown, the computing system 1000 includes, without limitation, a processor 1005, a network interface 1015, a memory 1020, and storage 1060, each connected to a bus 1017. The computing system 1000 may also include an input/output (I/O) device interface 1010 connecting I/O devices 1012 (e.g., keyboard, display and mouse devices) to the computing system 1000. More generally, any operating system supporting the functions disclosed herein may be used.

The processor 1005 retrieves and executes programming instructions stored in the memory 1020 as well as stored in the storage 1060. The bus 1017 is used to transmit programming instructions and application data between the processor 1005, I/O device interface 1010, storage 1060, network interface 1015, and memory 1020. Note, processor 1005 is included to be representative of a single processor, multiple processors, a single processor having multiple processing cores, and the like, and the memory 1020 is generally included to be representative of a random access memory. Illustratively, the memory 1020 includes an interworking component 1022, which is configured to implement method 900 of FIG. 9 and/or other techniques described herein. The storage 1060 may be a disk drive or flash storage device. Although shown as a single unit, the storage 1060 may be a combination of fixed and/or removable storage devices, such as fixed disc drives, removable memory cards, optical storage, network attached storage (NAS), or a storage area-network (SAN).

### EXAMPLE CLAUSES

Implementation examples are described in the following numbered clauses:
Clause 1: A system comprising: a laser system comprising a laser configured to emit electromagnetic radiation in laser pulses; and a first controller configured to: obtain an indication of an interworking control scheme for communicating with the laser system; receive inputs from an input device; and communicate with the laser system for control of the laser based on the inputs and according to the interworking control scheme.
Clause 2: The system of Clause 1, wherein the interworking control scheme comprises a message-based interworking control scheme.
Clause 3: The system of any one of Clauses 1-2, wherein to communicate with the laser system, the first controller is configured to: generate a message comprising the inputs; and transmit the message to the laser system.
Clause 4: The system of Clause 3, wherein the laser system comprises a second controller configured to: generate control signals based on the inputs within the message; and control operation of the laser based on the control signals.
Clause 5: The system of any one of Clauses 1-4, wherein the first controller is external to the laser system.
Clause 6: The system of Clause 1, wherein the interworking control scheme comprises an analog-based interworking control scheme.
Clause 7: The system of Clause 6, wherein to communicate with the laser system, the first controller is configured to: generate one or more control signals, based on the inputs; and transmit the one or more control signals to the laser system.
Clause 8: The system of Clause 7, wherein the one or more control signals comprise pulse width modulation signals.
Clause 9: The system of Clause 7, wherein the one or more control signals comprise serial peripheral interface signals.
Clause 10: The system of any one of Clauses 1-9, wherein the input device comprises an adjustable footswitch configured to actuate within subranges to communicate the inputs to the first controller.
Clause 11: A method comprising: obtaining an indication of an interworking control scheme for communicating with a laser system comprising a laser configured to emit electromagnetic radiation in laser pulses; receiving inputs from an input device; and communicating with the laser system for control of the laser based on the inputs and according to the interworking control scheme.
Clause 12: The method of Clause 11, wherein the interworking control scheme comprises a message-based interworking control scheme.
Clause 13: The method of any one of Clauses 11-12, wherein communicating with the laser system comprises: generating a message comprising the inputs; and transmitting the message to the laser system.
Clause 14: The method of Clause 13, wherein transmission of the message triggers an internal controller within the laser system to control operation of the laser based on the inputs within the message.
Clause 15: The method of Clause 11, wherein the interworking control scheme comprises an analog-based interworking control scheme.
Clause 16: The method of Clause 15, wherein communicating with the laser system comprises: generating one or more control signals, based on the inputs; and transmitting the one or more control signals to the laser system to control operation of the laser.
Clause 17: The method of Clause 16, wherein the one or more control signals comprise pulse width modulation signals.
Clause 18: The method of Clause 16, wherein the one or more control signals comprise serial peripheral interface signals.
Clause 19: The method of any one of Clauses 11-18, wherein the input device comprises an adjustable footswitch configured to actuate within subranges to communicate the inputs.
Clause 20: An apparatus comprising: one or more memories collectively storing instructions; and one or more processors coupled to the one or more memories, the one or more processors being collectively configured to execute the instructions to cause the apparatus to perform an operation comprising: obtaining an indication of an interworking control scheme for communicating with a laser system comprising a laser configured to emit electromagnetic radiation in laser pulses; receiving inputs from an input device; and communicating with the laser system for control of the laser based on the inputs and according to the interworking control scheme.
Clause 21: An apparatus comprising: one or more memories collectively storing instructions; and one or more processors coupled to the one or more memories, the one or more processors being collectively configured to execute the instructions to cause the apparatus to perform a method according to any one of Clauses 11-19.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a c c, b-b, b-b-b, b-b-c, c-c, and c-c-c or any other ordering of a, b, and c).

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

Within a claim, reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112(f) unless the element is expressly recited using the phrase “means for” or, in the case of a method claim, the element is recited using the phrase “step for.” The word “exemplary” is used herein to mean “serving as an example, instance, or illustration.” Any aspect described herein as “exemplary” is not necessarily to be construed as preferred or advantageous over other aspects.

What is claimed is:

1. A system comprising:
a laser system comprising a laser configured to emit electromagnetic radiation in laser pulses; and
a first controller configured to:
obtain an indication of an interworking control scheme for communicating with the laser system;
receive inputs from an input device; and
communicate with the laser system for control of the laser based on the inputs and according to the interworking control scheme.

2. The system of claim 1, wherein the interworking control scheme comprises a message-based interworking control scheme.

3. The system of claim 2, wherein to communicate with the laser system, the first controller is configured to:
generate a message comprising the inputs; and
transmit the message to the laser system.

4. The system of claim 3, wherein the laser system comprises a second controller configured to:
generate control signals based on the inputs within the message; and
control operation of the laser based on the control signals.

5. The system of claim 3, wherein the first controller is external to the laser system.

6. The system of claim 1, wherein the interworking control scheme comprises an analog-based interworking control scheme.

7. The system of claim 6, wherein to communicate with the laser system, the first controller is configured to:
generate one or more control signals, based on the inputs; and
transmit the one or more control signals to the laser system.

8. The system of claim 7, wherein the one or more control signals comprise pulse width modulation signals.

9. The system of claim 7, wherein the one or more control signals comprise serial peripheral interface signals.

10. The system of claim 1, wherein the input device comprises an adjustable footswitch configured to actuate within subranges to communicate the inputs to the first controller.

11. A method comprising:
obtaining an indication of an interworking control scheme for communicating with a laser system comprising a laser configured to emit electromagnetic radiation in laser pulses;
receiving inputs from an input device; and
communicating with the laser system for control of the laser based on the inputs and according to the interworking control scheme.

12. The method of claim 11, wherein the interworking control scheme comprises a message-based interworking control scheme.

13. The method of claim 12, wherein communicating with the laser system comprises:
generating a message comprising the inputs; and
transmitting the message to the laser system.

14. The method of claim 13, wherein transmission of the message triggers an internal controller within the laser system to control operation of the laser based on the inputs within the message.

15. The method of claim 11, wherein the interworking control scheme comprises an analog-based interworking control scheme.
